SEQUENCE LISTING (1 sequence)

SEQ ID NO: 1 - protein, 39 aa, synthetic construct
  SITE 2: note = Aib
  SITE 13: note = Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)1- CO-(CH2)18-CO2H
  MOD_RES 39: note = C-terminal amino acid is amidated as a C-terminal primary amide
YXEGTFTSDYSIXLDKIAQKAFVQWLIAGGPSSGAPPPS

(19) United States
(12) Patent Application Publication
(10) Pub. No.: US 2026/0035428 A1
(43) Pub. Date: Feb. 5, 2026

(54) A GIP/GLP1 FOR USE IN THERAPY

(71) Applicant: Eli Lilly and Company, Indianapolis, IN (US)

(72) Inventors: Brian David BENNYWORTH, Zionsville, IN (US); Elisa RAZZOLI, Indianapolis, IN (US)

(21) Appl. No.: 19/107,739

(22) PCT Filed: Sep. 11, 2023
(86) PCT No.: PCT/US2023/073817

§ 371 Date: Feb. 28, 2025

Related U.S. Application Data
(60) Provisional application No. 63/405,565, filed on Sep. 12, 2022.

(51) Int. Cl. C07K 14/575; A61K 38/00

(52) U.S. Cl. CPC C07K 14/575; A61K 38/00

(57) ABSTRACT

The present invention provides a method for improving glycemic control in a patient in need thereof, comprising administering to said patient, tirzepatide, or a pharmaceutically acceptable salt thereof, wherein said patient has failed to reach their HbA1c goal using GLP-1 RA treatment. The present invention provides a method for improving weight management in a patient in need thereof, comprising administering to said patient tirzepatide, or a pharmaceutically acceptable salt thereof, wherein said patient has failed to reach their weight management goal using GLP-1 RA treatment.

The present invention relates to the field of medicine. More particularly, the present invention provides new methods for improving glycemic control, in a patient in need thereof, wherein said patient has been unable to achieve their diabetes treatment goals using glucagon-like peptide-1 receptor agonist (GLP-1 RA).

Diabetes mellitus is a chronic disorder characterized by hyperglycemia resulting from defects in insulin secretion, insulin action, or both. In type 2 diabetes (T2D), the combined effects of impaired insulin secretion and insulin resistance are associated with elevated blood glucose levels. While therapy with currently approved doses of GLP-1 RA treatments enable the majority of patients to attain their glycemic targets (with or without use of other concomitant medications for T2D), significant numbers of patients receiving approved therapies today, including the GLP-1 RA treatments, liraglutide, semaglutide, and dulaglutide, are not reaching glycemic control goals (see, e.g., Stark Casagrande et al. The prevalence of meeting A1c, blood pressure, and LDL goals among people with diabetes, 1988-2010. Diabetes Care. 2013;36(8):2271-2279). Therefore, there remains an important medical need to provide enhanced efficacy of pharmaceutical agents while also preserving an overall acceptable benefit/risk profile.

A patient may be unable to achieve their HbA1c goal level despite receiving GLP-1 RA treatment. A patient may be in need of further chronic weight management when the patient receiving GLP-1 RA treatment fails to reach their target weight. The present invention provides a treatment option for such patients unable to achieve their HbA1c treatment goals despite using appropriate GLP-1 RA treatment. The present invention provides a treatment option for such patients unable to achieve their weight loss goals despite using appropriate GLP-1 RA treatment.

The approved label for tirzepatide requires initate dosing for four weeks using a once weekly 2.5 mg dose. Accordingly, several months of treatment may be required to achieve the desired dose for a particular patient. There is a desire for a dosing regimen providing for even more rapid achievement of the desired maximum dose, with acceptable patient comfort, and/or reduced gastrointestinal effects.

U.S. Pat. No. 9,474,780 generally describes compositions containing a GIP and GLP-1 receptor agonist administered by parenteral routes, and generally discloses a wide dosage range up to about 30 mg per person per week. U.S. Pat. No. 9,474,780 discloses the use of GIP/GLP1 co-agonists for treating diabetes, obesity, and other conditions. U.S. Pat. No. 9,474,780 describes and claims tirzepatide. Tirzepatide is approved by the U.S. Food and Drug Agency for use in treating type 2 diabetes, as an adjunct to diet and exercise.

Dulaglutide, the active ingredient in Trulicity®, is a GLP-1 RA that is approved for use as an adjunct to diet and exercise to improve glycemic control in patients with Type 2 diabetes (T2D). Dulaglutide has been used to treat many patients with T2D, resulting in significant reductions in HbA1c with low risk of hypoglycemia, and reductions in body weight. Dulaglutide is a well known GLP-1 RA.

Semaglutide, the active ingredient in Ozempic®, Rybelsis®, and Wegovy®, respectively, is a GLP-1 RA that is approved for use as an adjunct to diet and exercise to improve glycemic control in patients with T2D. Likewise, semaglutide is a well known GLP-1 RA.

Despite improvements provided by GLP-1 RA treatment, certain patients continue to be unable to attain their HbA1c treatment goals. There is a need for additional treatment options to provide desired glycemic control, as evidenced for example, by further reductions of HbA1c, and/or weight loss, while maintaining an acceptable profile of safety and adverse events. Also, there is a need for a treatment option for a patient unable to achieve their metabolic syndrome goals using a GLP-1 RA comprising administering tirzepatide for a condition selected from chronic kidney disease, atherosclerosis, NAFLD, and NASH.

In another embodiment, is a method for treating a patient unable to reach their HbA1c goal despite receiving GLP-1 RA treatment, comprising administering tirzepatide, or a pharmaceutically acceptable salt thereof. In an embodiment, tirzepatide is administered for at least 20 weeks. In an other embodiment, is a method for improving chronic weight management in a patient in need thereof, wherein said patient is unable to achieve their weight loss goal using a GLP-1 RA treatment, comprising administering tirzepatide, or a pharmaceutically acceptable salt thereof to a patient in need thereof. In certain embodiments, the GLP-1 RA is once weekly 0.75 mg dulaglutide. In certain embodiments, the GLP-1 RA is once weekly 1.5 mg dulaglutide. In certain embodiments, the GLP-1 RA is once weekly 3.0 mg dulaglutide. In certain embodiments, the GLP-1 RA is once weekly 4.5 mg dulaglutide. In certain embodiments, the GLP-1 RA is 0.75 mg dulaglutide. In certain embodiments, the GLP-1 RA is semaglutide. In an embodiment, a GLP-1 RA is once daily 7 mg oral semaglutide. In an embodiment, a GLP-1 RA is once daily 14 mg oral semaglutide. In certain embodiments, the GLP-1 RA is once weekly 1.0 mg semaglutide. In certain embodiments, the GLP-1 RA is once weekly 2.0 mg semaglutide. In certain embodiments GP1-RA is 2.4 mg semaglutide administered once weekly. In certain embodiments, GLP-1 RA is 1.4 mg liraglutide. In certain embodiments, GLP-1 RA is 1.8 mg liraglutide. In certain embodiments, a GLP-1 RA is administered within 1 week prior to initiation of tirzedpatide administration. In certain embodiments, a GLP-1 RA is administered within 2 weeks prior to initiation of tirzepatide administration. In certain embodiments, a GLP-1 RA is administered within 3 weeks of initiation of tirzepatide administration. In certain embodiments, a GLP-1 RA is administered within one month of initiation of tirzepatide administration.

Further, a once weekly tirzepatide dose may be administered for more than about four weeks. In certain embodiments, a tirzepatide dose may be increased to the next higher dose as contemplated herein, if additional glycemic control is needed. In certain embodiments, a tirzepatide dose is increased by 2.5 mg for at least 4 weeks. As used herein “tirzepatide initiation dose” is the first dose of tirzepatide administered to a patient in need thereof. A tirzepatide initiation dose in accordance with the dosing herein, may be once weekly 5 mg dose tirzepatide or a pharmaceutically acceptable salt thereof. A tirzepatide initiation dose may be once weekly 2.5 mg tirzepatide, or a pharmaceutically acceptable salt thereof.

In another aspect, the present invention provides a method of treating a patient unable to achieve their HbA1c goal despite treatment with a GLP-1 RA for at least 3 months, comprising: administering to said patient a once weekly tirzepatide dose of about 5.0 mg of tirzepatide, or a pharmaceutically acceptable salt thereof, for a minimum of about four weeks. In another aspect, the present invention provides a method of treating a patient with HbA1c between 6.5 to 9% despite treatment with a GLP-1 RA for at least 3 months, comprising: administering to said patient a once weekly tirzepatide dose of about 5.0 mg of tirzepatide, or a pharmaceutically acceptable salt thereof, for a minimum of about four weeks.

In another aspect, the present invention provides a method of treating a patient unable to achieve their HbA1c goal despite treatment with a GLP-1 RA for at least 6 months, comprising: administering to said patient a once weekly tirzepatide dose of about 5.0 mg of tirzepatide, or a pharmaceutically acceptable salt thereof, for a minimum of about four weeks.

In another aspect, the present invention provides a method of treating type 2 diabetes in a patient in need thereof, wherein said patient has been unable to achieve HbA1 goals despite treatment using a GLP-1 RA, comprising:
a) administering to said patient a once weekly tirzepatide dose of about 2.5 mg of tirzepatide, or a pharmaceutically acceptable salt thereof, for a minimum of about four weeks; and thereafter
a) administering to said patient a once weekly tirzepatide dose of about 5.0 mg of tirzepatide, or a pharmaceutically acceptable salt thereof, about once weekly for a minimum of about four weeks; and thereafter
b) administering to said patient a once weekly tirzepatide dose of about 7.5 mg of tirzepatide, or a pharmaceutically acceptable salt thereof, for a minimum of about four weeks; and thereafter
c) administering to said patient a once weekly tirzepatide dose of about 10.0 mg of tirzepatide, or a pharmaceutically acceptable salt thereof, for a minimum of about four weeks; and thereafter
d) administering to said patient a once weekly tirzepatide dose of about 12.5 mg of tirzepatide, or a pharmaceutically acceptable salt thereof, for a minimum of about four weeks; and thereafter
e) administering to said patient a once weekly tirzepatide dose of about 15.0 mg of tirzepatide, or a pharmaceutically acceptable salt thereof, about once weekly for a minimum of about four weeks.

In an aspect of the invention, dosing proceeds as described above, by the prior paragraphs; however, dosing once weekly 2.5 mg tirzepatide, or pharmaceutical salt thereof, is omitted, such that the initial tirzepatide dose is 5 mg once weekly.

Furthermore, the present invention provides a method of improving glycemic control in a patient in need thereof, wherein said patient is unable to achieve their HbA1c goal using GLP-1 RA, comprising:
a) administering to said patient a once weekly tirzepatide dose of about 2.5 mg of tirzepatide, or a pharmaceutically acceptable salt thereof, for a minimum of about four weeks; and thereafter
b) administering to said patient a once weekly tirzepatide dose of up to about 15.0 mg of tirzepatide, or a pharmaceutically acceptable salt thereof.

Another embodiment provides a method of improving glycemic control in a patient in need thereof, wherein said patient is unable to achieve HbA1c goals using GLP-1 RA, comprising:
a) administering to said patient a once weekly tirzepatide dose of about 5.0 mg of tirzepatide, or a pharmaceutically acceptable salt thereof, for a minimum of about four weeks.

Another embodiment provides a method of improving glycemic control in a patient in need thereof, wherein said patient is unable to achieve HbA1c goals using GLP-1 RA, comprising:
a) administering to said patient a once weekly tirzepatide dose of about 20.0 mg of tirzepatide, or a pharmaceutically acceptable salt thereof.

Another embodiment provides a method of improving glycemic control in a patient in need thereof, wherein said patient is unable to achieve HbA1c goals using GLP-1 RA, comprising:
a) administering to said patient a once weekly tirzepatide dose of about 25.0 mg of tirzepatide, or a pharmaceutically acceptable salt thereof.

In yet another aspect, the present invention provides a method of improving weight management in a patient in need thereof, wherein said patient is unable to achieve weight loss goals using GLP-1 RA, comprising:
a) administering to said patient a once weekly tirzepatide dose of about 2.5 mg of tirzepatide, or a pharmaceutically acceptable salt thereof, for a minimum of about four weeks; and thereafter
b) administering to said patient a once weekly tirzepatide dose of up to about 15.0 mg of tirzepatide, or a pharmaceutically acceptable salt thereof.

In yet another aspect, the present invention provides a method of improving weight management in a patient in need thereof, wherein said patient is unable to achieve weight loss goals using GLP-1 RA, comprising:
a) administering to said patient a once weekly tirzepatide dose of about 2.5 mg of tirzepatide, or a pharmaceutically acceptable salt thereof, for a minimum of about four weeks: and thereafter
b) administering to said patient a once weekly tirzepatide dose of up to about 20.0 mg of tirzepatide, or a pharmaceutically acceptable salt thereof.

In yet another aspect, the present invention provides a method of improving weight management in a patient in need thereof, wherein said patient is unable to achieve weight loss goals using GLP-1 RA, comprising:
a) administering to said patient a once weekly tirzepatide dose of about 2.5 mg of tirzepatide, or a pharmaceutically acceptable salt thereof, for a minimum of about four weeks; and thereafter
b) administering to said patient a once weekly tirzepatide dose of up to about 25.0 mg of tirzepatide, or a pharmaceutically acceptable salt thereof.

In yet another aspect, the present invention provides a method of improving weight management in a patient in need thereof, wherein said patient is unable to achieve weight loss goals using GLP-1 RA, comprising:
c) administering to said patient an initial once weekly tirzepatide dose of about 5.0 mg of tirzepatide, or a pharmaceutically acceptable salt thereof, for a minimum of about four weeks.

In another aspect, the present invention provides a method to cure diabetes, induce remission or prevent diabetes in a patient in need thereof, wherein said patient is unable to achieve such cure, remission, prevention using a GLP-1 RA, comprising: administering to said patient once weekly tirzepatide. In another aspect, the present invention provides a method to cure diabetes, induce remission or prevent diabetes in a patient in need thereof, wherein said patient is unable to achieve such cure, remission, prevention using a GLP-1 RA, comprising: administering to said patient a once weekly 5.0 mg of tirzepatide, or a pharmaceutically acceptable salt thereof.

The present invention provides a use of tirzepatide, or a pharmaceutically acceptable salt thereof, in the manufacture of a medicament for treating type 2 diabetes in a patient in need thereof, wherein said patient is unable to achieve type 2 diabetes goals using a GLP-1 RA, comprising:
a) administering to said patient once weekly tirzepatide.

In another aspect, the present invention provides a use of tirzepatide, or a pharmaceutically acceptable salt thereof, in the manufacture of a medicament for improving glycemic control in a patient in need thereof, wherein said patient is unable to achieve their HbA1c goal using a GLP-1 RA, comprising: administering to said patient once weekly tirzepatide.

Another embodiment provides a use of tirzepatide, or a pharmaceutically acceptable salt thereof, in the manufacture of a medicament for improving weight management in a patient in need thereof, wherein said patient has been unable to achieve their weight loss goal using dulaglutide, comprising:
a) administering to said patient a once weekly tirzepatide dose of about 2.5 mg of tirzepatide, or a pharmaceutically acceptable salt thereof, for a minimum of about four weeks; and thereafter
b) administering to said patient a once weekly tirzepatide dose of about 15.0 mg of tirzepatide, or a pharmaceutically acceptable salt thereof, for a minimum of about four weeks.

Another embodiment provides a use of tirzepatide, or a pharmaceutically acceptable salt thereof, in the manufacture of a medicament for improving weight management in a patient in need thereof, wherein said patient has been unable to achieve the patient weight loss goal using a GLP-1 RA treatment, comprising:
a) administering to said patient a once weekly tirzepatide dose of about 2.5 mg of tirzepatide, or a pharmaceutically acceptable salt thereof, for a minimum of about four weeks.

In another aspect, the present invention provides a use of tirzepatide, or a pharmaceutically acceptable salt thereof, in the manufacture of a medicament for improving weight management in a patient in need thereof, wherein said patient has been unable to achieve the patient weight loss goal using a GLP-1 RA treatment, comprising: administering an initial tirzepatide dose of about 5.0 mg of tirzepatide, or a pharmaceutically acceptable salt thereof.

Nonalcoholic fatty liver disease (“NAFLD”) is a liver disease characterized by an accumulation of fat in the liver of afflicted patients. Patients suffering from NAFLD may consume little or no alcohol, and in an embodiment, the patient does not have comorbid diabetes. NAFLD is a major cause of liver disease worldwide. Younossi et. al. Global epidemiology of nonalcoholic fatty liver disease-Meta-analytic assessment of prevalence, incidence, and outcomes; Hepatology (July 2016) 64:1; 73-84. Nonalcoholic steatohepatitis (“NASH”) is a type of NAFLD with an etiological constellation exhibiting macrovesicular hepatic steatosis, inflammation, hepatocyte ballooning, and fibrosis.

NAFLD and NASH are progressive diseases characterized by the development of liver fibrosis as NAFLD progresses to NASH.

In an embodiment, is a method for treating type 2 diabetes in a patient undergoing basal insulin treatment comprising administering an effective amount of tirzepatide for at least 4 weeks, and decreasing basal insulin dosage. In an embodiment is a method for treating type 2 diabetes in a patient diagnosed with type 2 diabetes for at least 5 years and currently receiving basal insulin treatment, comprising administering an effective amount of tirzepatide and decreasing basal insulin administration.

In a clinical study, patients with long standing type 2 diabetes already using basal insulin were randomized to tirzepatide (TZP) 5 mg, 10 mg or 15 mg for a 52 weeks treatment. The median dose of basal insulin at the time of treatment initiation was 46 U/day. At 52 weeks, patients on TZP had significant reduction in HbA1c (−1.92%, −2.15% and −2.3% for Tirzepatide 5 mg, 10 mg and 15 mg, respectively) and 58%, 71.9% and 72.8% of patients reached a HbA1c targe of <7%. Patients did not require to add any more basal insulin, rather substantial less use of basal insulin compared to treatment stat was noted. The median dose of basal insulin at 52 weeks was 20.3, 12.0 and 8.3 U/day, for TZP 5 mg, 10 mg and 15 mg, respectively. Additionally, 8%, 15% and 21% of patients were completely off of basal insulin at 52 weeks, for TZP 5 mg, 10 mg and 15 mg, respectively.

The present invention provides a method for treating NAFLD, comprising administering an effective amount of tirzepatide, or a pharmaceutically acceptable salt thereof, to a patient in need thereof, wherein said patient has been unable to resolve their NAFLD using a GLP-1 RA treatment. The present invention provides a method for treating NASH, comprising administering an effective amount of tirzepatide, or a pharmaceutically acceptable salt thereof, to a patient in need thereof, wherein said patient has been unable to resolve their NASH using a GLP-1 RA treatment.

Chronic kidney disease (“CKD”) is defined as abnormalities of kidney structure or function, present for three months, with implications for health of the patient.

The present invention provides a method for treating CKD comprising administering an effective amount of tirzepatide to a patient in need thereof, wherein said patient has been unable to resolve their CKD using a GLP-1 RA treatment.

U.S. Pat. No. 9,474,780 teaches that tirzepatide is useful for the treatment of diabetes, wherein “treating” includes restraining, slowing, stopping, or reversing the progression or severity of an existing symptom or disorder. Despite advances in the treatment of diabetes, many patients receiving such treatment are unable to reach their glycemic control goal or HbA1c goal. As used herein, the term “GLP-1 RA” means a glucagon-like peptide-1 receptor agonist treatment, wherein the pharmacological activity of the GLP-1 RA is primarily through agonism of a GLP1 receptor. For avoidance of doubt, the term GLP-1 RA does not embrace the dual GIP:GLP1 agonist, tirzepatide. In an embodiment, GLP-1 RA means a GLP-1 RA treatment approved for use in treating T2D by the United States FDA or corresponding regulatory body. For example, GLP-1 RA includes, but is not limited to, semaglutide, dulaglutide, liraglutide, as well as other oral and/or subcutaneous GLP-1 RA medicaments approved for administration in the treatment of diabetes and/or weight management. In certain embodiments, dulaglutide is a preferred GLP-1 RA. In certain embodiments, semaglutide is a preferred GLP-1 RA. In certain embodiments, liraglutide is a preferred GLP-1 RA. As used herein, it is contemplated that a GLP-1 RA will be administered in accordance with its respective FDA approved label dosing instructions.

In certain embodiments, tirzepatide is administered for at least four weeks. In certain embodiments, tirzepatide is administered for at least 20 weeks. In certain embodiments, tirzepatide is administered for at least 6 months. In certain embodiments, tirzepatide is administered for at least one year. In certain embodiments, GLP-1 RA is administered within one week of an initial tirzepatide dose. In certain embodiments, GLP-1 RA is co-administered with tirzepatide.

As used herein, the term “diabetes medication,” “diabetes medicine” and the like, means a medication approved by the pertinent regulatory agency for use in the treatment of glycemic control or Type II diabetes. In an embodiment, a patient continues administration of diabetes medicine, concommitant with administration of tirzepatide. In an embodiment, the patient maintains their HbA1c goal level for at least one month without further tirzepatide administration. In an embodiment, the patient who was unable to achieve their HbA1c goal using GLP-1 RA treatment, maintains their glycemic goal for at least 6 months after their last treatment with tirzepatide. In an embodiment, a tirzepatide dosage is administered within 2 weeks from the last GLP-1 RA dosage. In an embodiment, a tirzepatide dosage is administered within 1 week from the last GLP-1 RA dosage. In an embodiment, tirzepatide is administered within 4 days from the last GLP-1 RA dosage. In an embodiment, tirzepatide is administered concomitantly with a GLP-1 RA.

In an embodiment, tirzepatide is administered to a patient in need of treatment for an addictive behavior. In an embodiment, is a method for treating an addictive behavior, comprising administering an effective amount of tirzepatide to a patient in need thereof. The term “addictive behavior” means that the patient has a compulsive urge to use substance(s) or engage in behaviors. Such addictive behavior continues despite harmful consequences. The urge to use substances may include, but not limited to, alcohol, drugs, nicotine, and excessive food. The urge to engage in behaviors may include, but not limited to, promiscuity, gambling, and other undesired mood altering behaviors.

The tirzepatide doses of the present invention are likely to have specific concentrations of 5 mg/mL, 10 mg/mL, 15 mg/mL, 20 mg/mL, 25 mg/mL, 30 mg/mL, 40 mg/mL, and 50 mg/mL. Such compositions may be presented in a pre-filled syringe. Such pre-filled syringe may be useful for administering one half milliliter of such composition per patient per dose. The doses of the present invention are typically administered subcutaneously. The doses are typically administered using a pre-filled, disposable pen, reusable pen, or automatic pen injector. In an embodiment, the device is an automatic injection apparatus as claimed by U.S. Pat. No. 8,734,394.

As used herein, “tirzepatide” means a GIP/GLP1 dual agonist peptide as described in U.S. Pat. No. 9,474,780 and described by CAS Registry Number: 2023788-19-2. Tirzepatide is described in Example 1 of U.S. Pat. No. 9,474,780, with the following sequence:

YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)₂-(γGlu)₁-CO-(CH₂)₁₈-CO₂H; and the C-terminal amino acid is amidated as a C-terminal primary amide (SEQ ID NO: 1).

As used herein, the term “administering” means the administration by a nurse, health care provider, patient or any other individual including self-administration. As used herein, administration may include prescribing, dispensing, or assisting in any way with delivery, as conducted by a health care professional.

As used herein, “pharmaceutically acceptable salt” is well known to the skilled artisan. In an embodiment is a pharmaceutically acceptable salt that is a tirzepatide trifluoroacetate salt. In an embodiment is tirzepatide is administered as a non-salt.

As used herein, the term “biomarker”, means a laboratory measurement that reflects the activity of a disease process. Biomarkers may be used to diagnose a disease or condition, and usually quantitatively correlate (either directly or inversely) with disease progression. In the clinical trial setting, a biomarker is a measure of the effect of a specific treatment that may correlate with an actual clinical endpoint but does not necessarily have a precise relationship; that is, a biomarker is a substitute measure for a clinical endpoint.

As used herein, the terms “treatment,” “treat,” “treating,” and the like, mean to include slowing or attenuating the progression of a disease or disorder. The terms include to alleviate, ameliorate, or reduce one or more symptoms of a disorder or condition, even if the disorder or condition is not eliminated or if the progression is not slowed. As used herein, HbA1c goal is an HbA1c biomarker level to be achieved by the patient, determined by a physician by assessing patient status, age, comorbidities and the like.

As used herein “refractory diabetes” means that a patient, using GLP-1 RA treatment is unable to achieve their glycemic or HbA1c goal. The term may mean a patient using semaglutide is unable to achieve their glycemic or HbA1c goal and/or weight management goal. The term may mean that a patient using dulaglutide is unable to achieve their glycemic or HbA1c goal and/or weight management goal. In an embodiment, the term “improving glycemic control” may mean that the HbA1c decrease using the regimen disclosed herein is greater than a patient is expected to achieve using the tirzepatide T2D label dosing regimen. In an embodiment, the weight management achieved using the dosing regimen disclosed herein is greater than expected for a patient using the tirzepatide T2D label dosing regimen.

In an embodiment, a patient using a dosing regimen disclosed herein in the treatment of diabetes reaches at least their glycemic control treatment goal, and the treatment goal is maintained with cessation of treatment using tirzepatide and all other diabetes medication. In an embodiment, the patient glycemic goal is normoglycemia, or HbA1c less than about 5.9% HbA1c.

“Glycemic control” refers to the maintenance or reduction of a subject's HbA1c levels; “improv[ing]” glycemic control refers to reductions in HbA1c; and “in need of further” glycemic control refers to a need for reductions in HbA1c. In an embodiment “in need of further glycemic control” means that the patient has not achieved their HbA1c goal determined by their physician.

In an embodiment is a method of using a dosing regimen comprising treating a patient a GLP-1 RA and a GIP/GLP1 dual agonist peptide, tirzepatide, or a pharmaceutically acceptable salt thereof, in a novel dosing regimen to treat a patient unable to meet their treatment goals using a GLP-1 RA treatment.

As used herein “resolve” CKD means that the patient adequately achieves biomarker targets suggesting slowing of progression or improvement in degree of CKD, as determined by said patient's physician.

As used herein “resolve” NASH or “resolve” NAFLD means that the patient achieves biomarker targets suggesting improvement in, alleviation of, and/or slowing of progression of NASH or NAFLD, as determined by said patient's physician. In an embodiment, “resolve” NASH or NAFLD means that a liver biopsy indicates alleviation or slowing progression of the condition.

As used herein “weight management” refers to a reduction in body weight. In an embodiment, weight management means the management of obesity in an individual. As used herein, “improving” weight management refers to a reduction in body weight. In an embodiment, “in need of further weight management” or “in need of further weight loss” means that the patient has not achieved their weight loss goal determined by their physician for health of said patient. As used herein “unable to achieve their weight loss goal” means that further weight loss is necessary to achieve the weight loss goal determined by a health care provider. In an embodiment, “unable to achieve their weight loss goal” means a weight loss goal was attained; however, the weight loss was not adequately maintained at the goal weight determined by patient's health care provider.

As used herein “HbA1c” refers to glycated hemoglobin levels, which develop when hemoglobin joins with glucose in the blood. HbA1c levels are a commonly used measure of glycemic control in patients with diabetes, with decreased HbA1c levels generally indicating improved glycemic control. In the context of the methods of the present invention, the methods of the present invention result in a decrease in HbA1c. In certain embodiments, the HbA1c is decreased relative to the HbA1c levels expected from treatment using the FDA approved tirzepatide dosing regimen. In certain embodiments, the patient side effect experience is improved relative to average treatment experience using FDA approved tirzepatide dosing regimen.

As used herein “patient” or “patients” refers to a mammal in need of treatment for a condition or disorder. In an embodiment, the patient is a human with a disease or condition wherein the patient is unable to achieve their treatment goal. In an embodiment, a patient is unable to achieve said patient's HbA1c goal using a GLP-1 RA treatment. In an embodiment, a patient is unable to achieve said patient's weight loss goal using a GLP-1 RA treatment.

As used herein “diabetes treatment goal” is the desired HbA1c level and/or weight loss determined by a health care professional. As used herein, HbA1c goal means an HbA1c level to achieve as determined by said patient's physician. A treatment goal may vary from patient to patient based on physician assessment.

### Biomarkers

Clinically relevant biomarkers are measured in clinical studies to further support the use of tirzepatide for treating CKD in a patient unable to achieve such biomarker levels using a GLP-1 RA.

Biomarkers predictive of NAFLD are observed during clinical studies to demonstrate the beneficial effect of tirzepatide in the treatment of NAFLD, in a patient unable to achieve such beneficial effect using GLP-1 RA. Biomarkers predictive of NASH are observed during clinical studies to demonstrate the beneficial effect of tirzepatide in the treatment of NASH. HbA1c levels in tirzepatide treated patients reaching their glycemic control goals, and ceasing the use of diabetes medications, are measured during follow up to validate a diabetes cure in such patients.

### EXAMPLE 1

### Clinical Dosing Regimen

An open-label, single-arm, multicenter, multinational, Phase 4 study to assess the changes in glycemic control when switching from a stable dose of a GLP-1 RA directly to tirzepatide 5 mg in participants with T2D. The study will include 150 subjects.

This study includes a screening period and a 12-week treatment period.

The primary objective based on the efficacy estimand will be evaluated using the EAS dataset. The primary analysis model for HbA1c measurements over time will be an MMRM. The response variable of MMRM will be change in HbA1c values from baseline obtained at each scheduled postbaseline visit. The independent variables of the MMRM model are visit, and country as fixed effects and baseline HbA1c as covariates. Missing data will be addressed by the MMRM model. No explicit imputation methods for missing data will be employed. The P values obtained for the least square means for change from baseline will be used to determine the statistical significance to achieve the primary objective.

MMRM, as used herein means a mixed model for repeated measures.

Efficacy Analysis Set (EAS): This analysis set will be used to estimate the efficacy estimand for the primary objective.

Data obtained during the treatment period from the population excluding participants who were inadvertently enrolled, excluding data after permanent discontinuation of treatment or initiation of prohibited medication.

### Sequences

Tirzepatide
SEQ ID NO: 1
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)₂-(γGlu)₁-CO-(CH₂)₁₈-CO₂H; and the C-terminal amino acid is amidated as a C-terminal primary amide.

We claim:

1. A method of improving glycemic control in a patient in need thereof, wherein said patient is unable to achieve their HbA1c treatment goal with GLP-1 RA treatment, comprising: administering once weekly tirzepatide, or pharmaceutically acceptable salt thereof.

2. (canceled)

3. A method as claimed by claim 1 wherein the patient HbA1c goal is 7.0 or less.

4. A method as claimed by claim 1 wherein the patient HbA1c goal is 6.5 or less.

5. (canceled)

6. A method as claimed by claim 1 wherein the patient HbA1c goal is normoglycemic.

7. A method as claimed by any claim 1 wherein a tirzepatide once weekly dose is selected from the group consisting of 2.5 mg. 5.0 mg, 7.5 mg, 10 mg, 12.5 mg, 15 mg, 20 mg, and 25 mg tirzepatide, or a pharmaceutically acceptable salt thereof.

8. A method as claimed by claim 1 wherein a tirzepatide once weekly dose is selected from the group consisting of 2.5 mg, 5.0 mg, 7.5 mg, 10 mg, 12.5 mg, and 15 mg tirzepatide, or a pharmaceutically acceptable salt thereof.

9. (canceled)

10. A method as claimed by claim 1 wherein an initial tirzepatide dose is 5.0 mg tirzepatide or a pharmaceutically acceptable salt thereof.

11. A method as claimed by claim 10 wherein a once weekly tirzepatide dose is administered for at least 4 weeks.

12. A method as claimed by claim 1, wherein a GLP-1 RA is dulaglutide.

13. A method as claimed by claim 1 wherein a GLP-1 RA is 3.0 mg once weekly dulaglutide.

14. A method as claimed by claim 1 wherein a GLP-1 RA is 1.5 mg once weekly dulaglutide.

15. A method as claimed by claim 1 wherein a GLP-1 RA is 4.5 mg once weekly dulaglutide.

16. A method as claimed by claim 1 wherein a GLP-1 RA is semaglutide.

17. (canceled)

18. A method as claimed by claim 16 wherein a semaglutide once daily dose is 14 mg.

19. (canceled)

20. (canceled)

21. A method as claimed by claim 16 wherein a semaglutide once weekly dose is 2.4 mg.

22. (canceled)

23. A method as claimed by claim 1 wherein the GLP-1 RA is liraglutide.

24. (canceled)

25. (canceled)

26. A method as claimed by claim 1 wherein the patient was treated with semaglutide for a treatment period of at least 6 months prior to administering tirzepatide, or a pharmaceutically acceptable salt thereof.

27. A method as claimed by claim 1 wherein a GLP-1 RA is a nonpeptide GLP-1 RA.

28. (canceled)

29. (canceled)

30. A method as claimed by claim 1 wherein the patient has a BMI greater than or equal to 25 kilograms/square meter (kg/m²).

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. A method for improving chronic weight management in a patient in need thereof wherein said patient is unable to achieve their weight management treatment goal with GLP-1 RA treatment, comprising: administering once weekly tirzepatide, or pharmaceutically acceptable salt thereof.

88. A method as claimed by claim 31 wherein the initial once weekly tirzepatide dose is 5.0 mg tirzepatide or a pharmaceutically acceptable salt thereof.
